(19) United States
(12) Patent Application Publication
(10) Pub. No.: US 2015/0250418 A1
(43) Pub. Date: Sep. 10, 2015

(54) Optical Pulse Rate Monitor

(71) Applicant: ICON Health & Fitness, Inc., Logan, UT (US)

(72) Inventor: Darren C. Ashby, Richmond, UT (US)

(21) Appl. No.: 14/642,318

(22) Filed: Mar. 9, 2015

Related U.S. Application Data
(60) Provisional application No. 61/950,598, filed on Mar. 10, 2014.

(51) Int. Cl. A61B 5/00; A61B 5/0205

(52) U.S. Cl. CPC A61B 5/6803; A61B 5/0002; A61B 5/02055; A61B 5/721; A61B 5/7278; A61B 5/02444

(57) ABSTRACT

A monitoring system includes a sensing unit attachable to a body part, an optical detector oriented to measure an amount of ambient light from the body part, and a wireless transmitter to transmit data collected with the optical detector to a remote device.

### RELATED APPLICATIONS

This application claims priority to provisional Patent Application No. 61/950,598 titled “Optical Pulse Rate Monitor” filed Mar. 10, 2014.

### BACKGROUND

Heart rate monitors are used to track a person's heart rate in real time. As the heart pumps blood through the arteries of the body, blood is pushed to the capillaries where the blood exchanges oxygen and other compounds for carbon dioxide and waste products. From the capillaries, the blood is returned to the heart through veins. A person can manually check his or her heart rate by placing his or her fingers against an artery where the skin is relatively thin. As the heart pumps, the pressure and volume in the artery temporarily increases, and the person can feel this pulse with his or her fingers.

Often, when a user performs an exercise routine, the user's heart rate increases. However, the user may not want to increase his heart rate too high for any number of reasons, including maximizing efficiency or fat loss. As a result, the user may try to keep his or her heart rate within a healthy range. Due to the demands of the workout, a user cannot easily find his or her pulse to measure an accurate pulse count during the workout. To compensate, the user can use a heart rate monitor to determine the user's heart rate and display the heart rate to the user during the workout in real time so the user can keep his or her heart rate within the desired range.

One commercially available type of heart rate monitor includes an ear piece that can be attached to a user's ear lobe. As the heart pumps, the blood volume in the ear lobe varies, which can be recorded with a sensor clipped to the user's ear. The heart monitor includes an infrared optical emitter that emits infrared light into one side of the ear lobe while an infrared light detector positioned on the opposite side of the ear lobe receives the amount of light that passes through the ear lobe. The blood absorbs the infrared light emitted into the ear lobe more than the other tissues of the ear. As a result, the amount of light received by the detector changes as the blood volume in the ear lobe changes. Such heart rate monitors may include a wire to a power source to operate the monitor's light source.

One type of heart rate monitor is disclosed in U.S. Patent Publication No. 2007/0219457 issued to Chiu-hsiang Lo. In this reference, a wireless ear clips heart rate monitor has a sensor unit of the ear clips type that detects a user's heart rate, a signal processing unit that receives and processes the signal generated from the sensor unit, and a wireless signal transmitting unit that receives the signals from the signal processing unit and then transmits the signals out. The sensor unit detects a frequency of the change of blood density to derive the heart rate. Another type of heart rate monitor is described in U.S. Patent Publication No. 2011/0066056 issued to Chenghua Huang.

### SUMMARY

In a preferred embodiment of the invention, a monitoring system includes a sensing unit attachable to a body part, an optical detector oriented to measure an amount of ambient light from the body part, and a wireless transmitter to transmit data collected by the optical detector to a remote device.

One aspect of the invention that may be combined with one or more other aspects herein, a monitoring system includes a sensing unit attachable to a body part.

One aspect of the invention that may be combined with one or more other aspects herein, the monitoring system includes an optical detector oriented to measure an amount of ambient light from the body part.

One aspect of the invention that may be combined with one or more other aspects herein, the ambient light is infrared light emitted from the body part based on a temperature of the body part.

One aspect of the invention that may be combined with one or more other aspects herein, the ambient light is reflected visible light.

One aspect of the invention that may be combined with one or more other aspects herein, the optical detector is oriented to detect the amount of the ambient light within a range that depicts light fluctuations corresponding to blood circulation characteristics in the body part.

One aspect of the invention that may be combined with one or more other aspects herein, the monitoring system includes an attachment member shaped to be secured within a piercing of the body part.

One aspect of the invention that may be combined with one or more other aspects herein, the monitoring system includes a harvesting mechanism arranged to harvest energy from an energy source external to the monitoring system.

One aspect of the invention that may be combined with one or more other aspects herein, the harvesting mechanism includes a thermopile oriented to absorb heat from the body part.

One aspect of the invention that may be combined with one or more other aspects herein, the optical detector is in communication with a processor and memory.

One aspect of the invention that may be combined with one or more other aspects herein, the memory includes programmed instructions to further cause the processor to determine a heart rate associated with the body part based at least in part on communications from the optical detector.

One aspect of the invention that may be combined with one or more other aspects herein, the programmed instructions to further cause the processor to remove a motion artifact from the communications of the optical detector.

One aspect of the invention that may be combined with one or more other aspects herein, the processor and the optical detector are in wireless communication.

One aspect of the invention that may be combined with one or more other aspects herein, the monitoring system includes an accelerometer that measures a motion artifact representing a motion of the sensing unit when the sensing unit is moving.

One aspect of the invention that may be combined with one or more other aspects herein, the body part is an ear.

One aspect of the invention that may be combined with one or more other aspects herein, the optical detector is oriented to change an optical range based on changes to a surrounding environment.

One aspect of the invention that may be combined with one or more other aspects herein, the monitoring system includes a measurement duration of the optical detector is shorter than an intervening period between multiple measurement durations and the measurement durations are less than a microsecond.

One aspect of the invention that may be combined with one or more other aspects herein, a monitoring system includes a sensing unit attachable to an ear.

One aspect of the invention that may be combined with one or more other aspects herein, the monitoring system includes an optical detector oriented to measure an amount of ambient light from the ear within a range that depicts light fluctuations corresponding to blood circulation characteristics in the ear.

One aspect of the invention that may be combined with one or more other aspects herein, the monitoring system includes a harvesting mechanism arranged to harvest energy from an energy source external to the monitoring system.

One aspect of the invention that may be combined with one or more other aspects herein, the optical detector is in communication with a processor and memory that includes programmed instructions to cause the processor to determine a heart rate associated based at least in part on communications from the optical detector.

One aspect of the invention that may be combined with one or more other aspects herein, the ambient light is infrared light emitted from the ear based on a temperature of the ear.

One aspect of the invention that may be combined with one or more other aspects herein, the ambient light is reflected visible light.

One aspect of the invention that may be combined with one or more other aspects herein, the harvesting mechanism includes a thermopile oriented to absorb heat from the body part.

One aspect of the invention that may be combined with one or more other aspects herein, a monitoring system includes a sensing unit attachable to an ear.

One aspect of the invention that may be combined with one or more other aspects herein, the monitoring system includes an optical detector oriented to measure an amount of visible light reflected off of the ear within a range that depicts light fluctuations corresponding to blood circulation characteristics in the ear.

One aspect of the invention that may be combined with one or more other aspects herein, the monitoring system includes a harvesting mechanism arranged to harvest energy from an energy source external to the monitoring system.

One aspect of the invention that may be combined with one or more other aspects herein, the harvesting mechanism includes a thermopile oriented to absorb heat from the ear.

One aspect of the invention that may be combined with one or more other aspects herein, the optical detector is in communication with a processor and memory that includes programmed instructions to cause the processor to determine a heart rate associated with the ear based at least in part on communications from the optical detector.

One aspect of the invention that may be combined with one or more other aspects herein, the monitoring system includes an accelerometer that measures a motion artifact representing a motion of the sensing unit when the sensing unit is moving.

### BRIEF DESCRIPTION OF THE DRAWINGS

The accompanying drawings illustrate various embodiments of the present apparatus and are a part of the specification. The illustrated embodiments are merely examples of the present apparatus and do not limit the scope thereof.

FIG. 1 illustrates a perspective view of an example of a user wearing an earring in accordance with the present disclosure.

FIG. 2a illustrates a cross sectional view of an example of an earring in accordance with the present disclosure.

FIG. 2b illustrates a cross sectional view of an example of an earring in accordance with the present disclosure.

FIG. 3a illustrates a diagram of an example of a waveform representing measurements of reflected light in accordance with the present disclosure.

FIG. 3b illustrates a diagram of an example of a waveform representing measurements of emitted light in accordance with the present disclosure.

FIG. 4 illustrates a view of an example of a monitoring system in accordance with the present disclosure.

FIG. 5 illustrates a side view of an example of an earring in accordance with the present disclosure.

FIG. 6 illustrates a side view of an example of an earring in accordance with the present disclosure.

Throughout the drawings, identical reference numbers designate similar, but not necessarily identical, elements.

### DETAILED DESCRIPTION

The principles described in the present disclosure include a monitoring system with a sensing unit that has an attachment mechanism attachable to a body part of a user, such as an ear lobe. The sensing unit includes an optical detector oriented to measure an amount of ambient light from the body part. In some embodiments, the ambient light is visible light that is reflected off of the ear. The amount of light absorbed versus the amount of light reflected off of the ear changes with the blood volume in the ear. Thus, as the blood volume changes in the ear with the heart rate, the amount of reflected light fluctuates. These fluctuations can be detected to determine the heart rate. In other examples, the ambient light is infrared radiation that is naturally emitted from the ear. The infrared radiation varies with the temperature of the ear. The blood carries heat to the ear, so as the blood volume increases, the amount of infrared light emitted from the ear increases. Likewise, as the blood volume in the ear decreases, the amount of infrared light emitted from the changes. These fluctuations can also be detected.

The optical detector can collect the measurements for the amount light over a continuous time period or just for multiple time discrete periods. A timestamp can be associated with each time discrete period. The recorded signals and timestamps may be used to construct a waveform that represents the blood volume over time in the ear lobe. As an example, a full cycle of blood flow in the ear lobe can include the blood volume cycling from a minimum blood volume to a maximum blood volume and back to the minimum blood volume. The peaks or troughs of the waveform can correspond to the number of times that the heart beats. Thus, the waveform derived from time discrete or continuous measurements can be used to determine the heart rate.

Substantial power savings can be realized with the principles described herein because the sensing unit incorporated into the earring does not use an artificially powered light source. Rather, according to one embodiment, the power comes from the natural environment, such from visual light or infrared radiation emitted from the body.

With such low power consumption levels, the sensing unit can operate at power levels low enough that the sensing unit can harvest sufficient energy from the body heat of the body part to which it is attached. For example, a sensing unit attached to the ear or another body part may include a thermopile that converts a temperature differential into electrical energy. Generally, the greater the temperature differential, the greater the amount of electrical energy that can be produced. However, the temperature differential between the outer ear's skin and the ambient air surrounding the ear may be sufficient to produce enough energy, especially during a user's workout where the user's body temperature is elevated. In some examples, a portion of the sensing unit is inserted into a piercing of the ear. In such examples, the portion inserted into the ear may have an even greater temperature difference with the ambient air during the user's workout.

For the purposes of this disclosure, terms such as “front” used in reference to the ear refer to a side of the ear that also contains the tragus and antitragus. Likewise, for the purposes of this disclosure, terms such as “back” used with reference to the ear refer to a side of the ear that is opposite the front side.

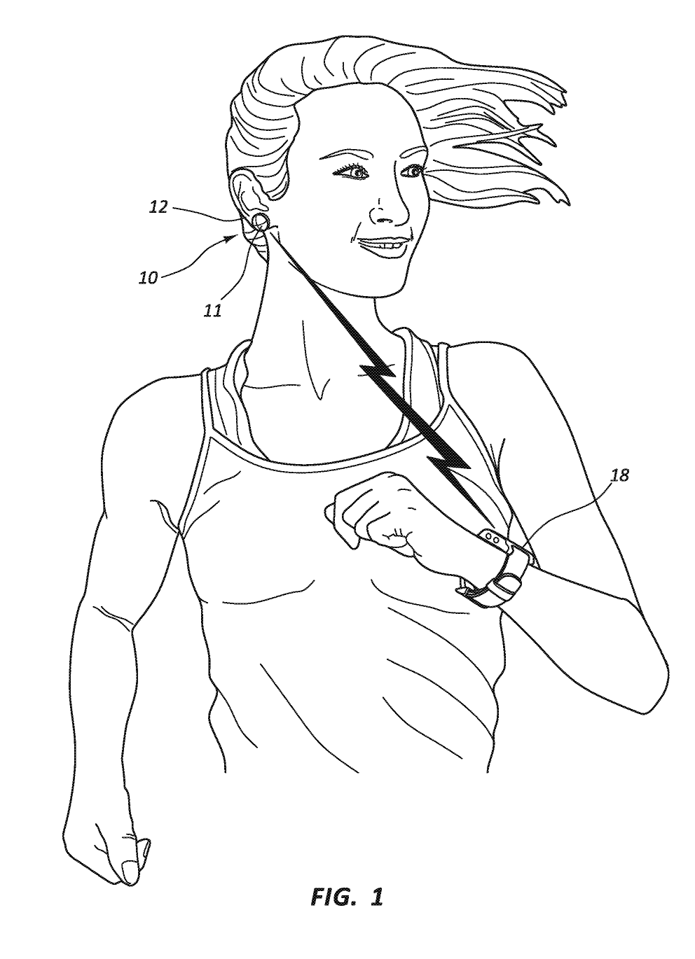

Particularly, with reference to the figures, FIG. 1 illustrates a perspective view of an example of a user wearing an earring 10. In this example, the earring 10 is attached to the user's ear lobe 12. A sensing unit 11 of the monitoring system 56 is incorporated into the earring 10. The sensing unit 11 of the monitoring system is incorporated into the earring 10. The sensing unit 11 includes a front portion 14 of the earring 10 exposed on a front side 16 of the ear lobe 12. The earring 10 is in communication with a remote device 18 where data collected from the earring 10 can be transmitted to the remote device 18.

The earring 10 may collect information about the user. For example, the user's heart rate may be determined based on measurements collected from the earring 10. These determinations may be calculated in the earring 10 and sent to the remote device 18 so that the data can be displayed to the user in real time. In other embodiments, the calculations occur at the remote device 18 or another device in communication with the remote device 18. For example, the measurements collected by the earring 10 are sent to the remote device 18 in raw form where the data can be processed. Some data processing may occur prior to the information being sent to the remote device 18. Such data processing may be used to lower the transmission time or to lower the transmission power from the earring 10 to the remote device 18.

In some embodiments, the earring 10 takes just heart rate measurements. For example, the optical detector 30 may record the amount of light transmitted through the ear lobe 12 and send this information to the remote device 18 by itself or with other types of recorded signals. The earring 10 may include an accelerometer 40, another type of sensor, or combinations thereof. The measurements from the accelerometer 40 or other sensors may be sent to the remote device 18 with the measurements from the optical detector 30. Information collected by the accelerometer 40 may be used to improve the heart rate calculations. For example, the motion of the user running may cause movements of the optical detector 30, which may skew the measurements. The accelerometer 40 can detect the movement of the user, the direction of the movement, the speed of the movement, and other types of information that may allow the earring 10, the remote device 18, or other device to correct for the motion artifacts in the measurements recorded by the optical detector 30.

The remote device 18 may be part of a mobile device that can perform the calculations to determine the user's heart rate. In other examples, the mobile device is incorporated into a treadmill, an elliptical, an stepper, a rowing machine, a stationary bike, cable exercise machine, or another type of exercise. For example, a user who is running on a treadmill may have an earring 10 that is in wireless communication with processing resources incorporated into the treadmill or other exercise machine. The signals may be obtained from the earring 10 by the treadmill, and the treadmill may determine a heart rate based on the obtained measurements. Further, the treadmill may present the heart rate to the user through a display incorporated into a control module. Additionally, the signals obtained from the earring 10 by the remote device may be used to determine other types of calculations, such as the number of calories consumed, an oxygen consumption amount, a blood pressure, cadence, distance, force, other types of information, or combinations thereof.

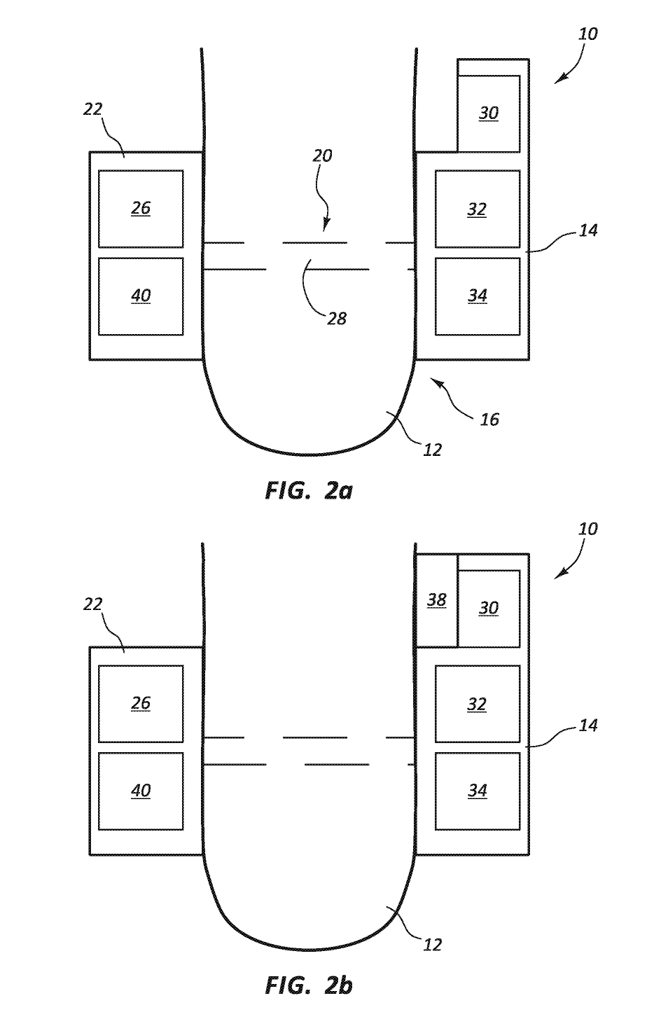

FIG. 2a illustrates a cross sectional view of an earring 10 in accordance with the present disclosure. In this example, the earring 10 includes an attachment member 20 that can be inserted into a piercing of the ear lobe 12. A back portion 22 of the earring 10 is connected to the attachment member 20. In some examples, the back portion 22 snaps on to the attachment member 20 or is otherwise attached to the attachment member 20. In other examples, the back portion 22 is integrally connected to the attachment member 20.

In the embodiment depicted in FIG. 2a, a power storage unit 26 provide powers to the components of the caning 10. A thermopile 28 is disposed within the attachment member 20 and is oriented to convert heat from the ear lobe 12 into electrical energy. In the illustrated example, the front portion 14 of the earring 10 includes the optical detector 30, memory 32, and a transmitter 34.

The thermopile 28 may be incorporated into the attachment member 20 such that the material of the attachment member 20 in direct contact with the ear lobe 12 absorbs heat from the ear lobe 12 and conducts the heat to a first side of the thermopile 28. The first side of the thermopile 28 has a first conductive material which contacts a second conductive material, which is dissimilar to the first conductive material. Collectively, the first and second conductive materials exhibit a Seebeck effect, which generates an electrical voltage when there is a temperature differential between the first and second conductive materials. The first conductive material receives the absorbed heat from the ear lobe 12 through the attachment member 20. In some examples, the attachment member 20 is made of the first conductive material. Also, the attachment member 20 may be a material that thermally conducts heat to the first conductive material. The second conductive material is in thermal communication with the ambient air around the user's ear. Thus, in situations where the ambient air is cooler than the ear lobe 12, a temperature differential exists and an electrical voltage is produced.

As a user exercises, the body generates excess heat. The excess heat is absorbed into the user's bloodstream, where the heat can be exchanged with a cooler temperature of the ambient environment around the user. Thus, as blood flows into the ear lobe 12 while the user is exercising, the excess heat from the body is collected by the attachment member 20, where the caning 10 can harvest the user's energy in the form of heat to run the sensing unit 11 with a harvesting mechanism, such a thermopile 28. To create a greater temperature differential, a heat sink and/or a heat spreader may be incorporated into the earring 10 to be in contact with the second conductive material.

Any appropriate material may be used in the thermopile 28 to convert the body's heat into to electrical energy. Examples of such materials may include, but are not limited to, chromel, constantan, iron, alumel, nickel, molybdenum, cobalt, nicrosil, nisil, copper, platinum, rhodium, tungsten, rhenium, gold, palladium, iridium, semiconductors, alloys thereof, mixtures thereof, or combinations thereof.

While this example has been described with specific reference to the harvesting mechanism being a device exhibits a Seebeck characteristic, other energy harvesting mechanisms can be used. For example, kinetic capture mechanisms, piezoelectric mechanisms, thermoelectric mechanism, other types of harvesting mechanisms, or combinations thereof can be used.

In the example of FIG. 2a, the electrical energy generated by the thermopile 28 is directed to a power storage unit 26. The power storage unit 26 may be a capacitor or other storage component that stores the electrical energy until the electrical energy is drawn out for use by the components of the earring. The stored electrical energy may be used to write a value recorded by the optical detector 30, transmit the values to the remote device 18, record an accelerometer reading, write values from the accelerometer reading, process the data recorded by the accelerometer 40 and/or optical detector 30, perform other functions, or combinations thereof. In other examples, chargeable batteries or other types of power storage units are incorporated into the earring 10 and are the recipients of the electrical energy from the thermopile 28. The batteries or other types of power storage units may be used to power the components of the earring 10.

In the illustrated example, the earring 10 forms a gap between the ear lobe and the location of the optical detector 30. As visible light contacts the ear lobe, a portion of the visible light is absorbed by the blood and tissues of the ear while another portion of the visible light is reflected off of the ear. The reflected portion of the visible light may be reflected towards the optical detector 30. In general, the blood within the ear lobe 12 absorbs more light than the other tissues within the ear. The other tissues in the ear have a consistent volume while the amount of blood in the ear is constantly changing based on the user's heart rate. As a result, less light may be reflected through the ear lobe 12 when the blood volume is higher, and more light may be reflected off of the ear lobe 12 when the blood volume is down.

In response to measuring an amount of ambient light, the optical detector 30 causes the value of the ambient light to be recorded in the memory 32. In alternative examples, in response to detecting the value of ambient light, the optical detector 30 automatically causes the value to be transmitted to the remote device 18.

The optical detector 30 may be a photodetector, which exhibits a photoelectric effect of converting light into electricity. In some examples, photodetectors are made of indium gallium arsenide. The photodetector may also be a semiconductor-based photodiode. Several types of photodiodes include p-n photodiodes, p-i-n photodiodes, and avalanche photodiodes. Metal-semiconductor-metal (MSM) photodetectors can also be used. In some cases, such optical-electrical converters can be coupled with a transimpedance amplifier and/or a limiting amplifier to produce a digital signal in the electrical domain from the incoming optical signal, which may be attenuated and distorted while passing through the ear lobe 12.

Any appropriate optical detector may be used in accordance with the principles described in the present disclosure. As non-limiting examples, the following types of optical detectors may be used: light emitting diodes that are reversed-biased to function as a photodiode; quantum devices that produce a discrete effect in response to detecting an individual photon; optical detectors that are effectively thermometers, responding purely to the heating effect of the incoming radiation, such as bolometers, pyroelectric detectors, Golay cells, other types of thermometers; photoresistors or Light Dependent Resistors (LDR) which change resistance in response to light intensity; photovoltaic cells that produce a voltage and supply an electric current when illuminated; photodiodes that can operate in a photovoltaic mode or a photoconductive mode; photomultiplier tubes containing a photocathode which emits electrons when illuminated; phototubes that contain a photocathode that emits electrons when illuminated; phototransistors that exhibit amplifying photodiode characteristics; quantum dot photoconductors or photodiodes that operate in the visible and infrared spectral regions; or combinations thereof.

In the example of FIG. 2a, memory 32 is in communication with the optical detector 30 and obtains the values of light intensity from the optical detector 30. The memory may be a buffer, a cache, or another type of memory that is programed to store the values from the optical detector 30 for a temporary amount of time. Generally, the storage time of the values in the memory 32 are long enough to store the information collected between transmission times. A transmitter 34 is in communication with the memory 32 and is programmed to send the values in the memory 32 to the remote device 18. In some examples, the transmitter 34 is positioned on the front portion 14 of the caning 10 to avoid having a transmission signal travel through the ear lobe 12 en route to the remote device 18. However, in other examples, the transmitter 34 is positioned into a back portion 22 of the earring 10.

An accelerometer 40 can also be incorporated into the earring 10. In the example of FIG. 2a, the accelerometer 40 is positioned in the back portion 22 of the earring 10; however, the accelerometer 40 may be positioned anywhere on the earring 10. The accelerometer 40 may sense motion of the earring 10 in multiple directions. Thus, as a user performs a workout, such as running, the movements of the user are picked up by the accelerometer 40. These measurements may be used to determine if a motion artifact exists in the values collected by the optical detector 30. If such a motion artifact exists, the values can be modified to reflect what the values would have without the motion artifact. The accelerometer's measurements may be sent to the memory 32 or directly to the transmitter 34 for conveyance to the remote device 18. In some embodiments, the accelerometer's measurements stay locally within the earring 10 and are used to modify the values from the optical detector 30 prior to sending the values to the remote device 18. In other examples, the calculations and other adjustments to be made based on the measurements from the accelerometer 40 are performed at the remote device 18.

FIG. 2b illustrates a cross sectional view of an earring 10 in accordance with the present disclosure. In this example, the naturally occurring infrared radiation from the ear is measured with the optical detector 30.

As the infrared radiation travels to the front side 16 of the ear lobe 12, the light enters a window 38 that is transparent to the radiation. The optical detector 30 may be positioned adjacent an optically transparent window 38 that is made of any appropriate material that is optically transparent to the radiation. The window is made of a material that is transparent or at least partially transparent to the infrared wavelengths being emitted. Examples of such windows may include arsenic trisulfide, barium fluoride, cadmium telluride, calcium fluoride, fused silica, gallium arsenide, germanium, polymers, led fluoride, lithium fluoride, magnesium fluoride, magnesium oxide, sapphire, sodium chloride, silicon, thallium bromo-iodide, zinc selenide, zinc sulfide, nanomaterials, crystalline materials, composites, other types of materials, or combinations thereof. In some examples, the window 38 is an optical waveguide that directs the emitted radiation towards the ear lobe 12.

While this example has been described with specific reference to incorporating a window, no window or waveguide may be used in other examples. Likewise, while the examples described in FIG. 2a describe measuring reflected light without a window, any appropriate window may be used to collect and/or direct light towards the optical detector 30.

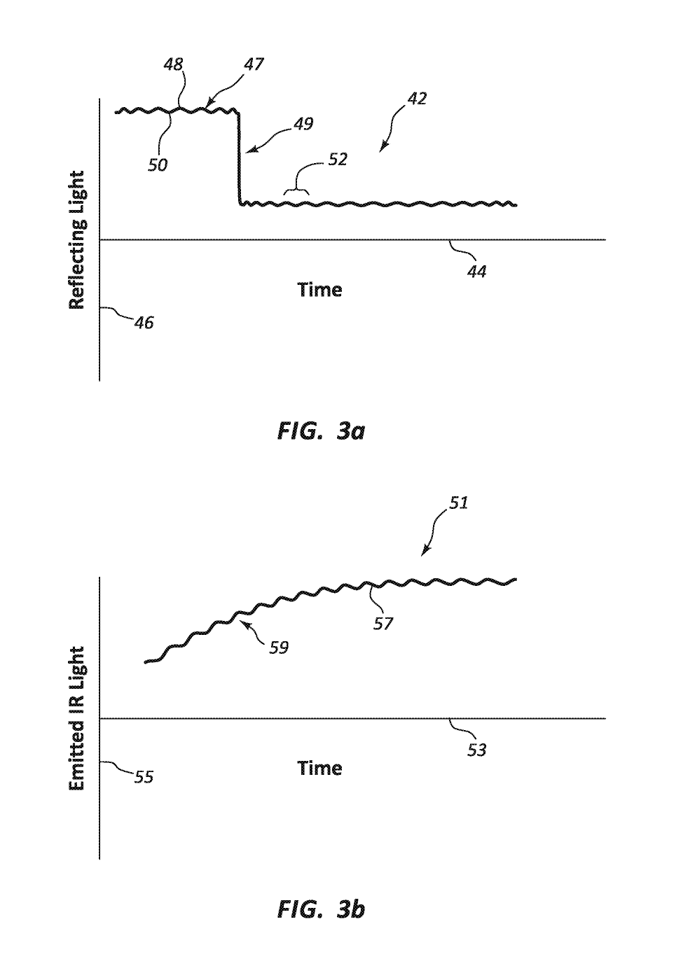

FIG. 3a illustrates a diagram of a waveform 42 representing measurements of reflected light in accordance with the present disclosure. In this example, the x-axis 44 represents time and the y-axis 46 represents measurements of reflected light. Line 47 represents the measurement of reflected light, which resembles a waveform that corresponds to the blood volume over time.

During a heartbeat, the muscles of the heart force an amount of blood into the arteries which causes a temporary surge of blood throughout the capillaries of the body, including at the ear lobe 12. As a result, the blood volume in the ear lobe 12 cycles between a high blood volume and a low blood volume. The peaks 48 of the waveform 42 represent the high blood volume generated as a result of the heartbeat. The troughs 50 of the waveform 42 represent the low volume between the heartbeats. One cycle 52 represents any point on the waveform 42 where the wave shape begins to approximately repeat itself. For example, a cycle 52 exists from one peak to the subsequent peak or from one trough to the subsequent trough. Each cycle represents a heartbeat. Thus, to determine the number of heartbeat, a processing element of the earring 10 or the remote device 18 can count the number of peaks 48, troughs 50, or other points in the waveform 42.

The heart rate can be calculated in the earring 10 or in the remote device 18. The determined heart rate can be presented to the user in any appropriate format. The heart rate may be presented in a display of a mobile device or in a display of a treadmill, elliptical, stepper, or other type of exercise machine. In yet other examples, the remote device 18 and/or earring 10 may audibly announce the heart rate. In addition to presenting the user his heart rate, the earring 10 and/or remote device 18 may also present information that is associated with the heart rate, such as oxygen consumption, calories burned, heart rhythm patterns, heart issues, warnings, other types of information, or combinations thereof.

Changes in the amount of reflected light may occur due to changes in the amount of light in the surrounding environment. Generally speaking, the amount of light in the surrounding environment may vary considerable throughout the user's workout. For example, the sun may be rising or setting during the workout. Further, the user may run through a shadow or a nearby street lamp may turn on. In the illustrated example, line 47 has a drop 49 that may occur when the user enters a shadow. The optical detector 30 may be capable of detecting just amounts of light within a specific range so that the fluctuations of reflected light within that range are more easily ascertainable. However, a significant change in the surrounding light that may occur from such a drop 49 may cause the measureable amount of light to fall outside of the detectable range. In such an example, the optical detector 30 may have the ability to auto adjust the range to go to a different range suitable to the amount of light that being reflected at that time.

FIG. 3b illustrates a diagram of another waveform 51 representing measurements of emitted light in accordance with the present disclosure. In this example, the x-axis 53 represents time, and the y-axis 55 represents emitted infrared light. Line 57 represents the amount of radiation measured.

The detected amount of infrared radiation may vary depending on at least two factors. One of these factors is the core temperature of the user. As the user progresses through his or her workout, the core temperature of the user may raise. This raise in temperature is depicted with the upward slope 59 during the initiation of the workout. In some cases, the user's core temperature stabilizes, which is depicted later in the workout as the line 57 levels off. Another factor that affects the amount of infrared radiation emitted from the ear is the amount of blood that is transported to the ear. The blood is generally heated by the user's core, so more infrared radiation is emitted when a fresh blood volume is pushed into the ear. As the blood volume cycles, the amount of the infrared radiation emitted from the ear fluctuates accordingly. These fluctuations are generally depicted with the peaks and troughs of the waveform 51 formed by line 57.

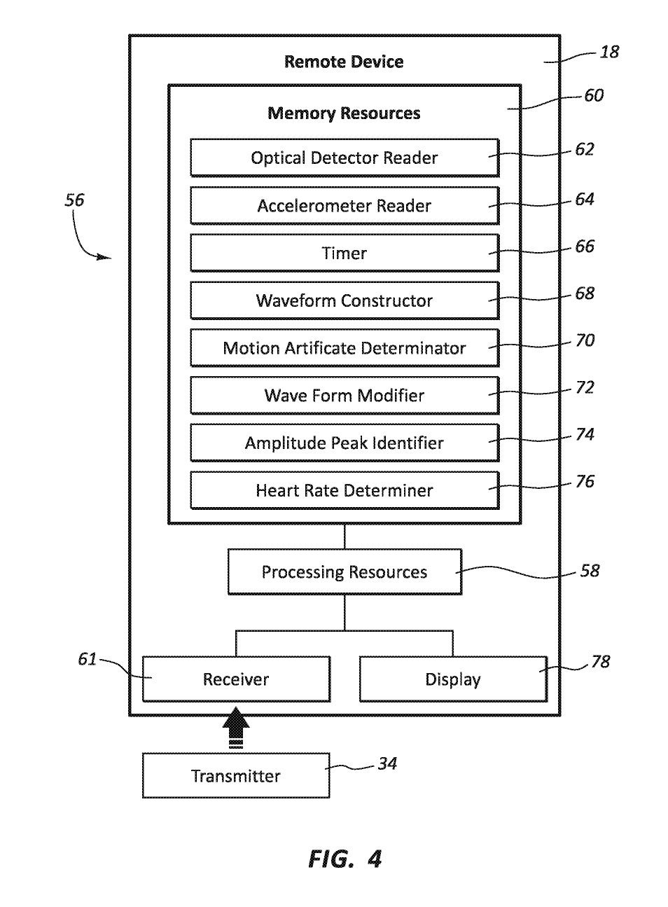

FIG. 4 illustrates a view of an example of a monitoring system 56 in accordance with the present disclosure. The monitoring system 56 may include a combination of hardware and program instructions for executing the functions of the monitoring system 56. In this example, the monitoring system 56 includes processing resources 58 that are in communication with memory resources 60. Processing resources 58 include at least one processor and other resources used to process programmed instructions. The memory resources 60 represent generally any memory capable of storing data such as programmed instructions or data structures used by the monitoring system 56. The programmed instructions shown stored in the memory resources 60 include an optical detector reader 62, an accelerometer reader 64, a timer 66, a waveform constructor 68, a motion artifact determiner 70, a waveform modifier 72, an amplitude peak identifier 74, and a heart rate determiner 76.

The memory resources 60 include a computer readable storage medium that contains computer readable program code to cause tasks to be executed by the processing resources 58. The computer readable storage medium may be tangible and/or non-transitory storage medium. The computer readable storage medium may be any appropriate storage medium that is not a transmission storage medium. A non-exhaustive list of computer readable storage medium types includes non-volatile memory, volatile memory, random access memory, write only memory, flash memory, electrically erasable program read only memory, magnetic storage media, other types of memory, or combinations thereof.

The optical detector reader 62 represents programmed instructions that, when executed, cause the processing resources 58 to read the values from the optical detector 30. The accelerometer reader 64 represents programmed instructions that, when executed, cause the processing resources 58 to read the values from the accelerometer 40. The signals from the transmitter 34 carrying optical detector and/or accelerometer measurements may be received with a receiver 61 in the remote device 18. The timer 66 represents programmed instructions that, when executed, cause the processing resources 58 to track the time that time discrete signals or continuous signals are sent and/or measured. In some examples, the timer 66 associates a time stamp with these signals.

The waveform constructor 68 represents programmed instructions that, when executed, cause the processing resources 58 to construct a waveform 42 that represents the blood volume in the ear lobe 12 based on the values recorded by the optical detector. The optical detector is capable of detecting light at discrete times. The measurement durations of the time discrete measurements are shorter than an intervening period between the measurement durations. As a result, the optical detector is off when appropriate, thereby saving additional amounts of power. In some examples, the intervening periods are at least twice as long as the measurement durations. Further, the measurement durations may last for a very short time periods. In some examples, the measurement duration is less than a microsecond. In other examples, the measurement duration is less than a nanosecond. Measurement durations in the picosecond or femtosecond range may also be used. Measurement durations in such short time intervals can result in the optical detector being off for the majority of time while still detecting and providing a sufficient amount of information to determine the user's heart rate.

A timestamp can be associated with each time discrete signals. The recorded signals and timestamps may be used to construct a waveform that represents the blood volume over time in the ear lobe. As an example, a full cycle of blood flow in the ear lobe can include the blood volume cycling from a minimum blood volume to a blood maximum volume and back to the blood minimum volume. The peaks (or troughs) of the waveform can correspond to the number of times that the heart beats. Thus, the waveform derived from the time discrete signals can be used to determine the heart rate.

A healthy heart rate at rest for a middle age adult is often between sixty and eighty beats per second. For well-trained athletes, the resting heart rates tend to be a little lower. However, the target heart rate range for most adults does not often exceed 170 beats per minute, and the estimated maximum heart rate for an adult is often less than 200. At 200 beats per minute, a full cycle of blood flow occurs every 0.3 seconds. At a sampling rate of 100 discrete signals per second, 30 discrete signal values can be used to construct a single cycle of the waveform. In examples where measurement duration is one nanosecond with a 100 samples taken a second, the optical detector is on for just 100 nanoseconds out of an entire second. In such an example, the optical detector is off for over 99.0 percent of the time. As a result, the optical detector consumes very little power.

The motion artifact determiner 70 represents programmed instructions that, when executed, cause the processing resources 58 to determine whether a motion artifact exists. If so, the motion artifact determiner 70 also determines the values of the motion artifact. The waveform modifier 72 represents programmed instructions that, when executed, cause the processing resources 58 to modify the waveform 42 based on the motion artifacts. The amplitude peak identifier 74 represents programmed instructions that, when executed, cause the processing resources 58 to identify the peaks 48 of the waveform 42. The heart rate determiner 76 represents programmed instructions that, when executed, cause the processing resources 58 to determine the heart rate based on the number of peaks 48 in the waveform 42 over time. The determined heart rate may be output to a display 78 where the heart rate can be presented to the user.

While this example has been described with reference to a specific mechanism for determining the heart rate based off of the output of the optical detector 30, any appropriate manner for determining the heart rate based on the optical detector's output may be used. For example, the heart rate may be determined with a different mechanism for determining the number of cycles in the waveform 42. Further, the values may be adjusted for the motion artifact before constructing the waveform 42. Other reasonable mechanism may also be used.

Further, the memory resources 60 may be part of an installation package. In response to installing the installation package, the programmed instructions of the memory resources 60 may be downloaded from the installation package's source, such as a portable medium, a server, a remote network location, another location, or combinations thereof. Portable memory media that are compatible with the principles described herein include DVDs, CDs, flash memory, portable disks, magnetic disks, optical disks, other forms of portable memory, or combinations thereof. In other examples, the program instructions are already installed. Here, the memory resources can include integrated memory such as a hard drive, a solid state hard drive, or the like.

The processing resources 58 and the memory resources 60 may be located within just the earring 10 or just the remote device 18. The memory resources 60 may be part of the earring's or the remote device's main memory, caches, registers, non-volatile memory, or elsewhere in the their memory hierarchy. Alternatively, the memory resources 60 may be in communication with the processing resources 58 over a network. Further, the data structures, such as libraries, may be accessed from a remote location over a network connection while the programmed instructions are located locally. Thus, the monitoring system 56 may be implemented with the earring, the remote device, other devices in communication with the earring and remote device, mobile devices, phones, wearable computing systems, other types of devices, or combinations thereof.

The monitoring system 56 of FIG. 4 may be part of a general purpose computer. However, in alternative examples, the monitoring system 56 is part of an application specific integrated circuit.

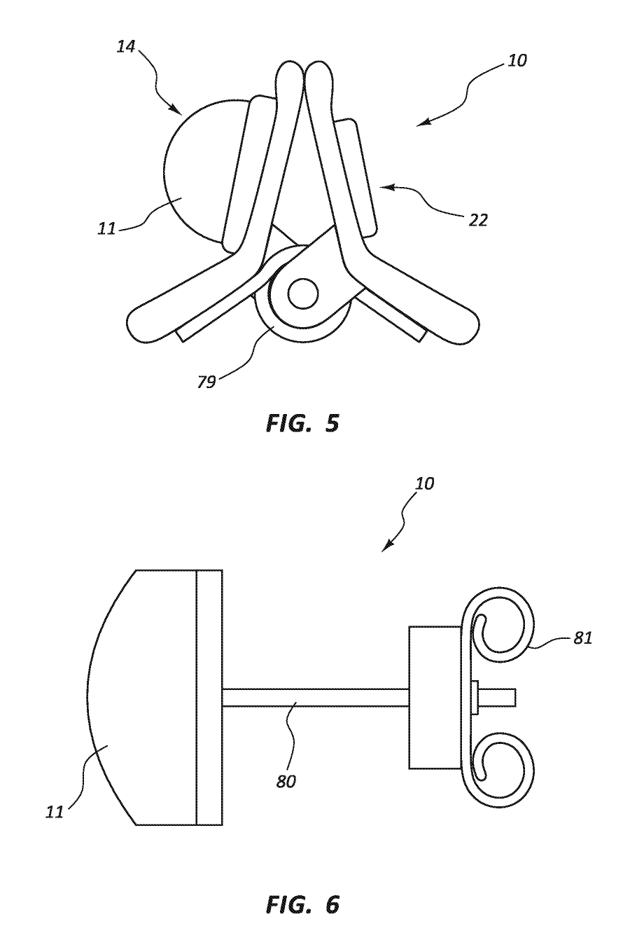

FIG. 5 illustrates a side view of an example of an earring 10 in accordance with the present disclosure. In this example, the earring 10 is attachable to the ear lobe 12 through compression. The front portion 14 and the back portion 22 are connected through a spring 79 that urges the front portion 14 and the back portion 22 together. The thermopile 28 and optical detector 30 may be integrated into the front or back portions 14, 22 of the earring 10 and may come into contact with the ear lobe through compression.

FIG. 6 illustrates a side view of an example of an earring 10 in accordance with the present disclosure. In this example, the earring 10 includes a stud 80 that is shaped to reside within a piercing. The optical detector 30, the thermopile 28, and the other components of the earring 10 may be incorporated into the stud 80, the backing 81, and/or the sensing unit 11. Further, a backing 81 to the earring 10 may include some of the earring's components, such as the transmitter 34 and accelerometer 40.

Further, while the examples above have been described with specific reference to using the thermal energy to power the components of the sensing unit, any appropriate mechanism for providing power to the components of the sensing unit may be used. For example, a kinetic capture mechanism may be used to convert kinetic energy into electrical energy to power the sensing unit. In other examples, a battery or another type of power source is integrated into the sensing unit.

### INDUSTRIAL APPLICABILITY

In general, the invention disclosed herein may convey heart rate information to a remote device while a user is exercising. Such a device may be incorporated into an earring, and thus may be convenient for users who already wear earrings while working out. However, any appropriate type of device may be used in accordance with the present disclosure. For example, the monitoring system may be incorporated into a hat, another piercing, a band, eye wear, hearing aid, another type of device that is attachable to a body part of a user, or combinations thereof.

The user can put the caning, earrings, or other type of device on before the workout. In examples where the monitoring device is an caning, the user puts on the earring just as he or she would do for other earrings that are conducive for working out. As the user begins to jog or otherwise perform the workout, a connection between the caning and the remote device may be established. The remote device may be a mobile device, a watch, a phone, a remote data base, or another type of remote device. In some cases, the remote device is a device strapped to or held by the person. In other cases, the remote device is a treadmill, an elliptical, or another device that facilitates a user's workout.

The connection may be initiated by the earring or the remote device. The remote device may detect that the monitoring device is within a proximity of the remote device and request to make a connection. In other examples, the monitoring device may broadcast a request to connect with the remote device. In response to the establishment of the connection, the earring may send heart rate information or another type of information to the remote device so that the heart rate information or other type of information can be determined and presented to the user.

The monitoring device may include a sensing unit that includes an optical detector. The optical detector may be oriented to measure an amount of ambient light from a user's body part that is proximate the sensing unit. For example, the user's body part may reflect ambient visible light from the environment in which the user is present, and the sensing unit may record that amount of light. In other examples, the user's body may detect infrared light emitted from the user's body. In examples where the sensing unit detects the infrared light, the amount of infrared light emitted from the body changes based on the amount of blood in the user's body part. For example, the sensing unit may detect more infrared light being emitted from a user's ear lobe when the ear lobe is filled with a greater amount of blood. The blood volume in the ear varies over time based on the heart rate. Thus, the heart rate can be determined based on the varying amounts of infrared light emitted from the body.

Likewise, in those embodiments where the visual light is reflected from the body part, more visual light may be absorbed depending on the blood volume in the body part. For example, the user's ear lobe may reflect a greater amount when there is a smaller blood volume in the ear lobe because the blood absorbs more visual light than the other tissues in the ear lobe. Thus, a smaller amount of visual light is detected when the blood volume is greater. As the blood volume varies over time, the amount of visual light reflected also varies. Thus, the sensing unit may report to the remote device a reliable parameter for determining the heart rate of the user. The changes in the reflected visual light and the emitted infrared light can match the changes in the ear's blood volume, which is based on the user's heart rate. As a result, the recorded changes in either reflected visual light or emitted infrared light correspond to the user's heart rate.

The remote device may use the recorded fluctuations in light to calculate a value of the heart rate. Such a heart value may be presented to the user in the remote device or another device. For example, where the remote device is a smart watch, the smart watch may present the heart rate value to the user as the user exercises. In other examples, the heart rate value may be determined by a smart phone which can also present the heart rate to the user. In some examples, the remote device is a smart phone which receives information directly from the monitoring device. The smart phone may determine the heart value and transmit that value to the smart watch where the heart rate value can be conveniently presented to the user. In some cases, the heart rate value may be transmitted to a database where the heart rate value can be retrieved at a later time. Such a database may be incorporated into the remote device, like a smart watch. However, in other examples, the database may be included in a data center, be associated with a website, another location, or combinations thereof. The heart rate value may be stored with other values that represent the distance that the user ran, the speed at which the user ran, the altitude of the workout, the location of the workout, the weather conditions of the workout, the time of day, the amount of food recently digested by the user, other types of information related to the user's workout, or combinations thereof.

With the computations occurring in the remote device, the processing power needed in the sensing unit/monitoring device can be reduced. The earring may be capable of using such a low amount of power that the differential of the user's body heat and the temperature in the ambient environment is great enough to provide a sufficient amount of electrical energy to the power the device. These temperature differences may be used to generate electrical power to drive the operations of the monitoring device with a thermopile, a Seebeck device, a Peltier device, another type of device, or combinations thereof. These types of devices can reduce or eliminate the batteries in the earring or other type of monitoring device and thus reduce the weight of the earring. By reducing the weight of the earring, the inertia and pull on the ear or other body part is reduced, which makes wearing the earring or other monitoring device during exercise more comfortable.

The monitoring device may be attached to the user in such a manner that is comfortable for the user to wear. For example, the principles described herein provide an effective mechanism for keeping the earring attached during exercise, because the earring has an attachment member that is inserted into a piercing. A backing of the earring can also reinforce the attachment of the earring to the ear. Such a piercing may already be used by the user to wear other types of jewelry when the user is not working out. However, the user is likely to remove certain types of jewelry before exercise anyway. In such an instance, the user may remove the other types of jewelry and insert the monitoring device. The piercing provides secure feature already in place in the user to secure the monitoring unit. In examples where the piercing is in the ear lobe, the user can wear the monitoring unit without having to wear another band or mechanism to hold another type of monitoring unit.

In some cases, the user may use a single earring or a single monitoring unit to track his or her heart rate. In other examples, the user can include multiple earrings or multiple monitoring units that are equipped to monitor the user's heart rate. For example, the monitoring unit may be incorporated into earrings for both ears or multiple places on the body. In such configurations, the earrings or other devices with sensing units can work together. For example, one of the sensing units in the earrings can operate while the other is inactive. While in an inactive state, the sensing unit can charge its power source through an energy harvesting mechanism as described above. Thus, if the energy harvesting mechanism fails to provide a consistent level of power over time of the sensing unit, the sensing unit can take periodic breaks to build up the power supply. In some examples, sensing units in two earrings may take alternating turns to monitor the heart rate. In other examples, sensing units in two earrings and a nose ring take turns monitoring the heart rate. In yet other examples, more than one of the sensing units can provide heart rate monitoring information to the remote device. In such cases, the measurements may be average or further processed to refine the heart rate determinations. In yet additional embodiments, each of the devices with sensing units can provide different types of information. For example, a first earring may include the optical detector and the second earring may include an accelerometer.

In some examples, an accelerometer may be incorporated into the monitoring unit to detect the movements that are experienced by the sensing unit. In some cases, the accelerometer may establish that the movements experienced by the monitoring unit were sufficiently large enough to generate a motion artifacts that skews the measured parameters. The measurements taken with the accelerometer may also be sent to the remote device where a value of the motion artifact is generated. The remote device may modify the heart rate values to reflect the motion artifacts and thereby improve the accuracy of the heart rate values.

The monitoring device may be any appropriate size. For example, the monitoring unit may have a length and width that are less than an inch. In other examples, at least one of the length or width of the monitoring unit is less than half an inch. Further, the monitoring device may weight any appropriate amount. In some examples, the weight of the monitoring device is small enough that the monitoring device does not put undue strain on the user's due to the monitoring unit's weight. For example, the monitoring device may weight about the same amount as commercial available earrings that are used as jewelry. The weight of the monitoring unit may be less than 15.0 grams, less than 10.0 grams, less than 7.0 grams, less than 5.0 grams, less than 4.0 grams, less than 3.0 grams, less than 2.0 grams, or less than 1.0 gram.

Further, the monitoring device may include any appropriate type of material. For example, the monitoring device may be made, in part, of plastic, gold, silver, metal, bronze, cobalt, stainless steel, titanium, sterling silver, glass, niobium, rubber, silicon, quartz, wood, polyester, materials commonly used in making earrings, another type of material, or combinations thereof.

Any appropriate type of earring structure may be used in accordance with the principles described herein. The earring types may include an ear cuff, stud earring, hoops earrings, dangle earrings, huggie earrings, other types of earrings, or combinations thereof. Further, while the above examples have been described with reference to attaching to the ear lobe, the earrings may be attached to any portion of the ear. For example, the earring may be attached to the ear lobe, the tragus, the anti-tragus, the helix, the anti-helix, the cartilage, the inner conch, the outer conch, the scapha, other portions of the ear, or combinations thereof. In some embodiments, the sensing unit is attached to other body parts. For example, the sensing unit may be attached to the nose, the mouth, navel, other body part, or combinations thereof.

The optical detector may be positioned adjacent an optically transparent window that is made of any appropriate material that is optically transparent to the radiation. The window is made of a material that is transparent or at least partially transparent to the infrared wavelengths being emitted. Examples of such windows may include arsenic trisulfide, barium fluoride, cadmium telluride, calcium fluoride, fused silica, gallium arsenide, germanium, polymers, led fluoride, lithium fluoride, magnesium fluoride, magnesium oxide, sapphire, sodium chloride, silicon, thallium bromo-iodide, zinc selenide, zinc sulfide, nanomaterials, crystalline materials, composites, other types of materials, or combinations thereof. In some examples, the window is an optical waveguide that directs the emitted radiation towards the ear lobe.

The optical detector may be a photodetector, which exhibits a photoelectric effect of converting light into electricity. In some examples, photodetectors are made of indium gallium arsenide. The photodetector may also be a semiconductor-based photodiode. Several types of photodiodes include p-n photodiodes, p-i-n photodiodes, and avalanche photodiodes. Metal-semiconductor-metal (MSM) photodetectors can also be used. In some cases, such optical-electrical converters can be coupled with a transimpedance amplifier and/or a limiting amplifier to produce a digital signal in the electrical domain from the incoming optical signal, which may be attenuated and distorted while passing through the ear lobe.

Any appropriate optical detector may be used in accordance with the principles described in the present disclosure. As non-limiting examples, the following types of optical detectors may be used: light emitting diodes that are reversed-biased to function as a photodiode; quantum devices that produce a discrete effect in response to detecting an individual photon; optical detectors that are effectively thermometers, responding purely to the heating effect of the incoming radiation, such as bolometers, pyroelectric detectors, Golay cells, other types of thermometers; photoresistors or Light Dependent Resistors (LDR) which change resistance in response to light intensity; photovoltaic cells that produce a voltage and supply an electric current when illuminated; photodiodes that can operate in a photovoltaic mode or a photoconductive mode; photomultiplier tubes containing a photocathode which emits electrons when illuminated; phototubes that contain a photocathode that emits electrons when illuminated; phototransistors that exhibit amplifying photodiode characteristics; quantum dot photoconductors or photodiodes that operate in the visible and infrared spectral regions; or combinations thereof.

Any appropriate type of energy harvesting mechanism may be used in accordance with the principles described in the present disclosure. The excess heat of the user may escape from the user's body through the ear lobe while the user is exercising. Such excess heat may be collected by the attachment member and harvested to run the sensing unit. In such examples, the attachment member may include a thermopile. Any appropriate material may be used in the thermopile to convert the body's heat into to electrical energy. Examples of such materials may include, but are not limited to, chromel, constantan, iron, alumel, nickel, molybdenum, cobalt, nicrosil, nisil, copper, platinum, rhodium, tungsten, rhenium, gold, palladium, iridium, semiconductors, alloys thereof, mixtures thereof, or combinations thereof. While these examples have been described with reference to the harvesting mechanism exhibiting Seebeck characteristics, other energy harvesting mechanisms can be used. For example, kinetic capture mechanisms, piezoelectric mechanisms, thermoelectric mechanism, other types of harvesting mechanisms, or combinations thereof can be used.

What is claimed is:

1. A monitoring system, comprising:
a sensing unit attachable to a body part; and
an optical detector oriented to measure an amount of ambient light from the body part; and
a wireless transmitter to transmit data collected by the optical detector to a remote device.

2. The monitoring system of claim 1, wherein the ambient light is infrared light emitted from the body part based on a temperature of the body part.

3. The monitoring system of claim 1, wherein the ambient light is reflected visible light.

4. The monitoring system of claim 1, wherein the optical detector is oriented to detect the amount of the ambient light within a range that depicts light fluctuations corresponding to blood circulation characteristics in the body part.

5. The monitoring system of claim 1, further comprising an attachment member shaped to be secured within a piercing of the body part.

6. The monitoring system of claim 1, further comprising a harvesting mechanism arranged to harvest energy from an energy source external to the monitoring system.

7. The monitoring system of claim 6, wherein the harvesting mechanism includes a thermopile oriented to absorb heat from the body part.

8. The monitoring system of claim 1, wherein the optical detector is in communication with a processor and memory.

9. The monitoring system of claim 8, wherein the memory includes programmed instructions to further cause the processor to determine a heart rate associated with the body part based at least in part on communications from the optical detector.

10. The monitoring system of claim 9, wherein the programmed instructions to further cause the processor to remove a motion artifact from the communications of the optical detector.

11. The monitoring system of claim 8, wherein the processor and the optical detector are in wireless communication.

12. The monitoring system of claim 1, further including an accelerometer measures a motion artifact representing a motion of the sensing unit when the sensing unit is in motion.

13. The monitoring system of claim 1, wherein the body part is an ear.

14. The monitoring system of claim 1, wherein the optical detector is oriented to change an optical range based on changes to a surrounding environment.

15. The monitoring system of claim 1, wherein a measurement duration of the optical detector is shorter than an intervening period between multiple measurement durations and the measurement durations are less than a microsecond.

16. A monitoring system, comprising:
a sensing unit attachable to an ear;
an optical detector oriented to measure an amount of ambient light from the ear within a range that depicts light fluctuations corresponding to blood circulation characteristics in the ear;
a harvesting mechanism arranged to harvest energy from an energy source external to the monitoring system; and
the optical detector is in communication with a processor and memory that includes programmed instructions to cause the processor to determine a heart rate associated based at least in part on communications from the optical detector.

17. The monitoring system of claim 16, wherein the ambient light is infrared light emitted from the ear based on a temperature of the ear.

18. The monitoring system of claim 16, wherein the ambient light is reflected visible light.

19. The monitoring system of claim 18, wherein the harvesting mechanism includes a thermopile oriented to absorb heat from the body part.

20. A monitoring system, comprising:
a sensing unit attachable to an ear;
an optical detector oriented to measure an amount of visible light reflected off of the ear within a range that depicts light fluctuations corresponding to blood circulation characteristics in the ear;
a harvesting mechanism arranged to harvest energy from an energy source external to the monitoring system;
the harvesting mechanism includes a thermopile oriented to absorb heat from the ear;
the optical detector is in communication with a processor and memory that includes programmed instructions to cause the processor to determine a heart rate associated with the ear based at least in part on communications from the optical detector; and
an accelerometer measures a motion artifact representing a motion of the sensing unit when the sensing unit is in motion.
